(19) United States
(12) Patent Application Publication
(10) Pub. No.: US 2020/0375675 A1
(43) Pub. Date: Dec. 3, 2020

(54) COMPLIANT END-EFFECTOR FOR IMAGE GUIDED SURGICAL PROCEDURES

(71) Applicant: KONINKLIJKE PHILIPS N.V., EINDHOVEN (NL)

(72) Inventors: Alexandru PATRICIU, BELMONT, MA (US); Aleksandra POPOVIC, BOSTON, MA (US)

(21) Appl. No.: 16/955,839

(22) PCT Filed: Dec. 14, 2018
(86) PCT No.: PCT/EP2018/084906

§ 371 Date: Jun. 19, 2020

Related U.S. Application Data
(60) Provisional application No. 62/608,604, filed on Dec. 21, 2017.

(51) Int. Cl. A61B 34/00; A61B 34/30; A61B 90/11; B25J 9/02; B25J 9/10; B25J 15/02

(52) U.S. Cl. CPC A61B 34/70; A61B 34/30; A61B 90/11; A61B 2034/303; B25J 9/10; B25J 15/02; A61B 2034/301; B25J 9/02

(57) ABSTRACT

A compliant medical robot system (50) employing a medical robot (50) and a robot actuation controller (80). The medical robot (50) includes a compliant end-effector (70) adjoined to a robotic arm (60), and the compliant end-effector (70) includes one or more tool actuators (72) adjoined to a tool holder (71) to provide a manual positioning of the tool holder (71) relative to the robotic arm (60). In operation, the robot actuation controller (80) controls an actuation of the tool actuator(s) (72) delineating an actuation parameter to set a manual positionable range of the tool holder (71) relative to the robotic arm (60) in compliance with a tool positioning command specifying the manual positionable range of the tool holder (71) relative to the robotic arm (60).

### CROSS REFERENCE TO RELATED APPLICATIONS

This application is the U.S. National Phase application under 35 U.S.C. § 371 of International Application No. PCT/EP2018/084906 filed Dec. 14, 2018, published as WO 2019/121378 on Jun. 27, 2019, which claims the benefit of U.S. Provisional Patent Application No. 62/608,604 filed Dec. 21, 2017. These applications are hereby incorporated by reference herein.

### FIELD OF THE INVENTION

The present disclosure generally relates to medical robot systems for performing various medical procedures (e.g., laparoscopic surgery, neurosurgery, spinal surgery, natural orifice transluminal surgery, cardiology, pulmonary/bronchoscopy surgery, biopsy, ablation, and diagnostic interventions). The present disclosure specifically relates to a compliant end-effector facilitating adjustment by a clinician of a trajectory of a medical tool into an anatomical region as the clinician manually positions the medical tool in a direction towards a target location within the anatomical region.

### BACKGROUND OF THE INVENTION

In robotic assisted image guided interventions, an insertion direction of a medical tool is provided by a medical robot. More particularly, the medical robot will provide a steady trajectory for a medical tool to be inserted by a clinician within an anatomical region and the medical tool will have only a linear translation in a direction towards a target location within the anatomical region. However, in some cases the clinician may need to make small adjustments to the tool alignment at the insertion time. These adjustments may be due to the fact that the target moved with respiration of the patient, or during the insertion the clinician “feels” an obstacle and desired to avoid that obstacle.

For example, as shown in FIG. 1A, a pedicle screw replacement intervention may involve a robotic arm 30 being actuated to robotically position an end-effector 31 relative to a pedicle 10 to provide a trajectory for a pedicle feeler 20 whereby pedicle feeler 20 may be held by end-effector 31 along a trajectory in a direction of a target cancellous bone channel 11 to a center of pedicle 10 and operated to drill a cylindrical hole along the trajectory leading target cancellous bone channel 11 to a center of pedicle 10. Currently, for any tool alignment corrections of the trajectory of pedicle feeler 20 necessary to reach the center of pedicle 10, robotic arm 30 may need to re-actuated within a limited motion range 32 defined by a limited spatial area 12 of cancellous bone channel 11. Such re-actuation of robotic arm 30 typically adds undesirably time to the procedure length and may decrease the accuracy of the procedure.

Also by example, as shown by FIG. 1B, a kidney biopsy intervention may involve robotic arm 30 being actuated to position end-effector 31 relative to a kidney 13 to provide a trajectory for a biopsy needle 21 to potentially cancerous cells 14 whereby biopsy needle 21 may be held by end-effector 31 along the trajectory to cells 14. However, kidney 30 may move due to a cyclical respiration 15 of kidney 30, which may require robotic arm 30 to be re-actuated within a limited motion range 33 to thereby chase cells 14. Again, such re-actuation of robotic arm 30 typically adds undesirably time to the procedure length and may decrease the accuracy of the procedure.

### SUMMARY OF THE INVENTION

The present disclosure describes a compliant medical robot system, controllers and methods incorporating a compliant end-effector for implementing a compliance profile delineating a positionable range of a tool holder relative to a robotic arm to thereby facilitate a manual positioning by a clinician of an alignment of a trajectory of a medical tool established by the tool holder to a target location within anatomical region. The compliance profile is based on a particular anatomy region that is to be traversed by the medical tool such that the accuracy and safety of the procedure is increased and the time for the procedure is decreased.

A first embodiment of the inventions of the present disclosure is a compliant medical robot system employing a medical robot and a robot actuation controller. The medical robot includes a compliant end-effector adjoined to a robotic arm, and the compliant end-effector includes one or more tool actuators adjoined to a tool holder to provide a manual positioning of the tool holder relative to the robotic arm. In operation, the robot actuation controller controls an actuation of the tool actuator(s) based on a compliance profile delineating an actuation parameter to set a manual positionable range of the tool holder relative to the robotic arm in compliance with a tool positioning command specifying the manual positionable range of the tool holder relative to the robotic arm.

A second embodiment of the inventions of the present disclosure is the robot actuation controller employing a global positioner and a tool positioner. In operation, the global positioner controls an actuation of the arm actuator(s) to robotically position the compliant end-effector in compliance with the end-effector positioning command specifying a position of the end-effector, and the target positioner controls an actuation of the tool actuator(s) based on the compliance profile delineating the actuation parameter to set a manual positionable range of the tool holder relative to the robotic arm in compliance with a tool positioning command specifying the manual positionable range of the tool holder relative to the robotic arm.

A third embodiment of the inventions of the present disclosure is a compliant medical robot control method involving the robot actuation controller controlling an actuation of the arm actuator(s) to robotically position the compliant end-effector in compliance with the end-effector positioning command specifying a position of the end-effector, and further involving the robot actuation controller controlling an actuation of the tool actuator(s) based on the compliance profile delineating the actuation parameter to set a manual positionable range of the tool holder relative to the robotic arm in compliance with the tool positioning command specifying the manual positionable range of the tool holder relative to the robotic arm.

For purposes of describing and claiming the inventions of the present disclosure:

(1) terms of the art of the present disclosure including, but not limited to, “robotic arm”, “end-effector” and “tool holder”, are to be broadly interpreted as known in the art of the present disclosure and exemplary described in the present disclosure;

(2) the term “medical procedure” broadly encompasses all diagnostic, surgical and interventional procedures, as known in the art of the present disclosure or hereinafter conceived, for an imaging, a diagnosis and/or a treatment of a patient anatomy;

(3) the term “medical tool” broadly encompasses, as understood in the art of the present disclosure and hereinafter conceived, a tool, an instrument, a device or the like for conducting an imaging, a diagnosis and/or a treatment of a patient anatomy. Examples of a medical tool include, but are not limited to, guidewires, catheters, scalpels, cauterizers, ablation devices, balloons, stents, endografts, atherectomy devices, clips, needles, forceps, k-wires and associated drivers, endoscopes, ultrasound probes, X-ray devices, awls, screwdrivers, osteotomes, chisels, mallets, curettes, clamps, forceps, periosteomes and j-needles;

(4) the term “adjoined” and any tense thereof broadly encompasses a detachable or a permanent coupling, connection, affixation, clamping, mounting, etc. of components;

(5) the term “actuator” broadly encompasses, all devices and mechanisms, as known in the art of the present disclosure and hereinafter conceived, utilized to effect motion and/or to maintain a position of one or more components of a robotic arm and/or an end-effector as exemplary described in the present disclosure;

(6) the descriptive labels for term “actuator” herein facilitates a distinction between actuators as described and claimed herein without specifying or implying any additional limitation to the term “actuator”;

(7) the term “compliant end-effector” broadly encompasses all end-effectors, as known in the art of the present disclosure and hereinafter conceived, incorporating one or more actuators for providing a manual positioning of a tool holder in accordance with the inventive principles of the present disclosure as exemplary described in the present disclosure;

(8) the term “medical robotic system” broadly encompasses all robotic systems, as known in the art of the present disclosure and hereinafter conceived, incorporating a robotic arm for supporting a translation, a rotation, and/or pivoting of an end-effector to thereby position the end-effector as pre-operatively and/or intra-operatively planned for a medical procedure. Examples of medical robotic systems include, but is not limited to, serial articulated robot arms employed by the da Vinci® Robotic System, the Medrobotics Flex® Robotic System, the Magellan™ Robotic System, and the CorePath® Robotic System;

(9) the term “compliant medical robotic system” broadly encompasses all medical robotic systems incorporating a compliant end-effector of the present disclosure as exemplary described in the present disclosure;

(10) the term “controller” broadly encompasses all structural configurations, as understood in the art of the present disclosure and as exemplary described in the present disclosure, of an application specific main board or an application specific integrated circuit for controlling an application of various inventive principles of the present disclosure as subsequently described in the present disclosure. The structural configuration of the controller may include, but is not limited to, processor(s), computer-usable/computer readable storage medium(s), an operating system, application module(s), peripheral device controller(s), slot(s) and port(s). A controller may be housed within or linked to a workstation. Examples of a “workstation” include, but are not limited to, an assembly of one or more computing devices, a display/monitor, and one or more input devices (e.g., a keyboard, joysticks and mouse) in the form of a standalone computing system, a client computer of a server system, a desktop or a tablet;

(11) the descriptive labels for term “controller” herein facilitates a distinction between controllers as described and claimed herein without specifying or implying any additional limitation to the term “controller”;

(12) the term “application module” broadly encompasses an application incorporated within or accessible by a controller consisting of an electronic circuit and/or an executable program (e.g., executable software stored on non-transitory computer readable medium(s) and/or firmware) for executing a specific application;

(13) the terms “signal”, “data” and “command” broadly encompasses all forms of a detectable physical quantity or impulse (e.g., voltage, current, or magnetic field strength) as understood in the art of the present disclosure and as exemplary described in the present disclosure for transmitting information and/or instructions in support of applying various inventive principles of the present disclosure as subsequently described in the present disclosure. Signal/data/command communication various components of the present disclosure may involve any communication method as known in the art of the present disclosure including, but not limited to, signal/data/command transmission/reception over any type of wired or wireless datalink and a reading of signal/data/commands uploaded to a computer-usable/computer readable storage medium; and

(14) the descriptive labels for terms “signal”, “data” and “commands” herein facilitates a distinction between signals/data/commands as described and claimed herein without specifying or implying any additional limitation to the terms “signal”, “data” and “command”.

The foregoing embodiments and other embodiments of the inventions of the present disclosure as well as various features and advantages of the present disclosure will become further apparent from the following detailed description of various embodiments of the inventions of the present disclosure read in conjunction with the accompanying drawings. The detailed description and drawings are merely illustrative of the inventions of the present disclosure rather than limiting, the scope of the inventions of present disclosure being defined by the appended claims and equivalents thereof.

### BRIEF DESCRIPTION OF THE DRAWINGS

FIG. 1 illustrates a pedicle screw replacement intervention as known in the art of the present disclosure.

FIG. 2 illustrates a kidney biopsy intervention as known in the art of the present disclosure.

FIG. 3 illustrates an exemplary embodiment of a compliant medical robotic system in accordance with the inventive principles of the present disclosure.

FIG. 4A illustrates a top XY view of an exemplary embodiment of a compliant end-effector incorporating pneumatic pistons in accordance with the inventive principles of the present disclosure.

FIG. 4B illustrates a bottom XY view of the exemplary embodiment of a compliant end-effector incorporating pneumatic pistons in accordance with the inventive principles of the present disclosure.

FIG. 4C illustrates a side XZ view of the exemplary embodiment of a compliant end-effector incorporating pneumatic pistons in accordance with the inventive principles of the present disclosure.

FIG. 4D illustrates a side YZ view of the exemplary embodiment of a compliant end-effector incorporating pneumatic pistons in accordance with the inventive principles of the present disclosure.

FIG. 5A illustrates a top XY view of an exemplary embodiment of a compliant end-effector incorporating diaphragms in accordance with the inventive principles of the present disclosure.

FIG. 5B illustrates a bottom XY view of the exemplary embodiment of a compliant end-effector incorporating diaphragms in accordance with the inventive principles of the present disclosure.

FIG. 5C illustrates a side XZ view of the exemplary embodiment of a compliant end-effector incorporating diaphragms in accordance with the inventive principles of the present disclosure.

FIG. 5D illustrates a side YZ view of the exemplary embodiment of a compliant end-effector incorporating diaphragms in accordance with the inventive principles of the present disclosure.

FIG. 6 illustrates an exemplary embodiment of an image guided medical procedure system in accordance with the inventive principles of the present disclosure.

FIG. 7 illustrates a pedicle screw replacement intervention in accordance with the inventive principles of the present disclosure.

FIG. 8 illustrates a kidney biopsy intervention in accordance with the inventive principles of the present disclosure.

### DETAILED DESCRIPTION OF THE PREFERRED EMBODIMENTS

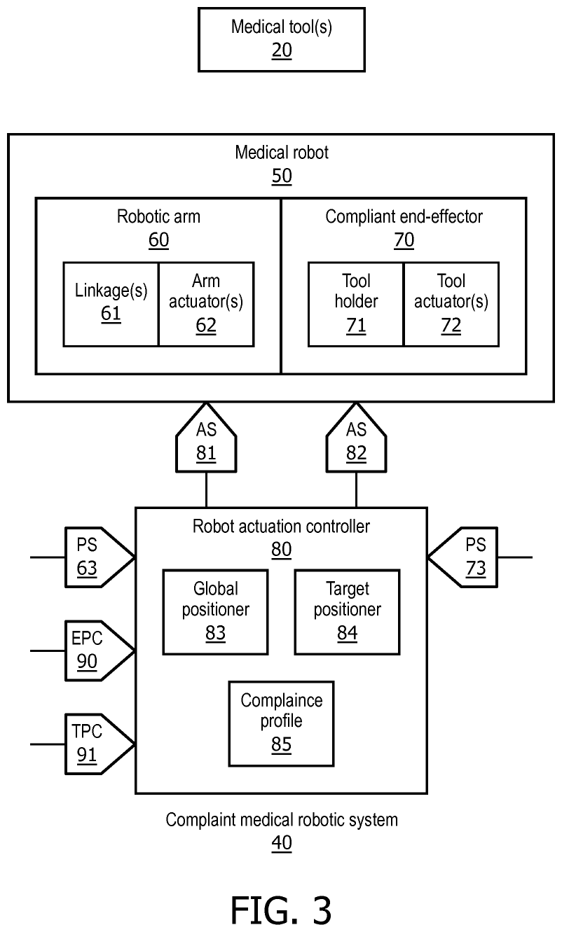

To facilitate an understanding of the inventions of the present disclosure, the following description of FIGS. 3-5 teach basic inventive principles of an exemplary embodiment of a compliant medical robotic system 40 of the present disclosure. From this description, those having ordinary skill in the art will appreciate how to apply the inventive principles of the present disclosure for making and using numerous and varied additional embodiments of a compliant medical robotic system of the present disclosure.

Referring to FIG. 3, compliant medical robotic system 40 of the present disclosure provides robotic guidance for one or more medical tools 20 utilized to conduct an imaging, a diagnosis and/or a treatment of a patient anatomy in accordance with a medical procedure as known in the art of the present disclosure. Examples of a medical tool 20 include, but are not limited to, needles, pedicle finders, j-needles, awls.

In practice, the robotic guidance of a medical tool 20 is dependent upon the particular medical procedure. Examples of such robotic guidance include, but are not limited to, interventional medical image guidance, preoperative medical image guidance, and preoperative plan guidance.

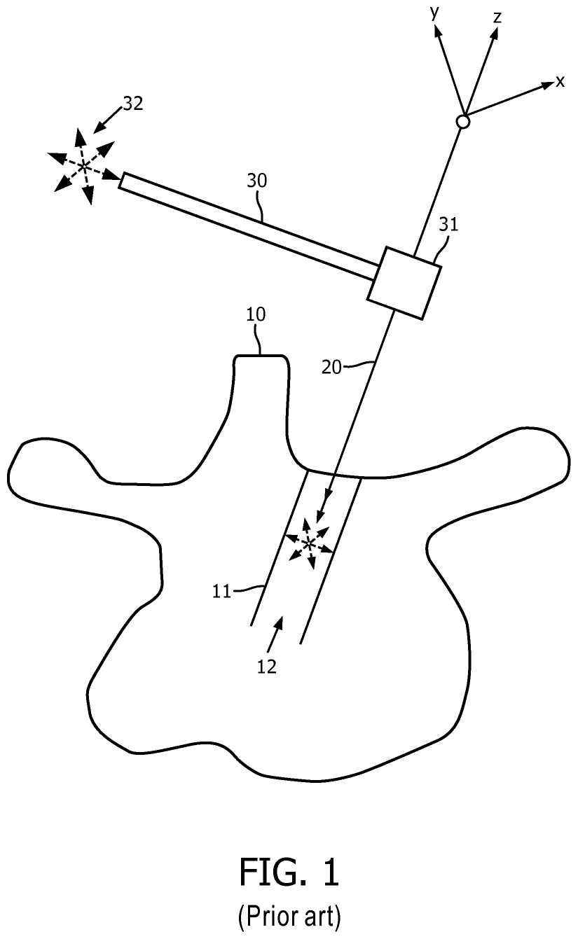

Still referring to FIG. 1, compliant medical robotic system 40 employs a medical robot 50 and a robot actuation controller 80.

Medical robot 50 includes a robotic arm 60 for robotically positioning a medical tool 20 within a medical procedural space (e.g., an operating room, a training room, etc.) as will be further described in the present disclosure.

In practice, robotic arm 60 may be configured in any component arrangement as known in the art of the present disclosure suitable for a medical procedure. In one embodiment as known in the art, robotic arm 60 includes one or more linkages 61, and one or more arm actuators 62 adjoined to linkage(s) for effecting motion and/or maintaining a position of linkage(s) 61.

Arm actuator(s) 62 is(are) actuatable by robot actuation controller 80 via actuation signal(s) 81 for controlling a pose of linkage(s) 61 as known in the art of the present disclosure, and arm actuator(s) 62 may include a pose sensor of any type (e.g., an encoder) for generating pose signal(s) 63 informative of a pose (i.e., orientation and/or location) of linkage(s) 61 relative to a reference as known in the art of the present disclosure.

In practice, an arm actuator 62 may be incorporated into any type of actuator joint controllable as known in the art of the present disclosure for effecting motion and/or maintaining a position of linkage(s) 61 including, but not limited to, a translational actuator joint, a ball and socket actuator joint, a hinge actuator joint, a condyloid actuator joint, a saddle actuator joint and a rotary actuator joint.

Medical robot 50 includes a compliant end-effector 70 for a manual positioning a medical tool 20 within a medical procedural space (e.g., an operating room, a training room, etc.) as will be further described in the present disclosure.

In accordance with the present disclosure, compliant end-effector 70 includes a tool holder 71 and one or more tool actuators 72 adjoined to tool holder 71 for effecting motion and/or maintaining a position of tool holder 71.

In practice, tool holder 71 may have any configuration as known in the art of the present disclosure for defining a trajectory for medical tools 20, and tool actuator(s) 72 may be incorporated into any type of actuator joint suitable controllable as known in the art of the present disclosure for effecting motion and/or maintaining a position of the trajectory of tool holder 71 including, but not limited to, a pressure actuator having a pressure setting controllable for effecting motion and/or maintaining a position of the trajectory of tool holder 71.

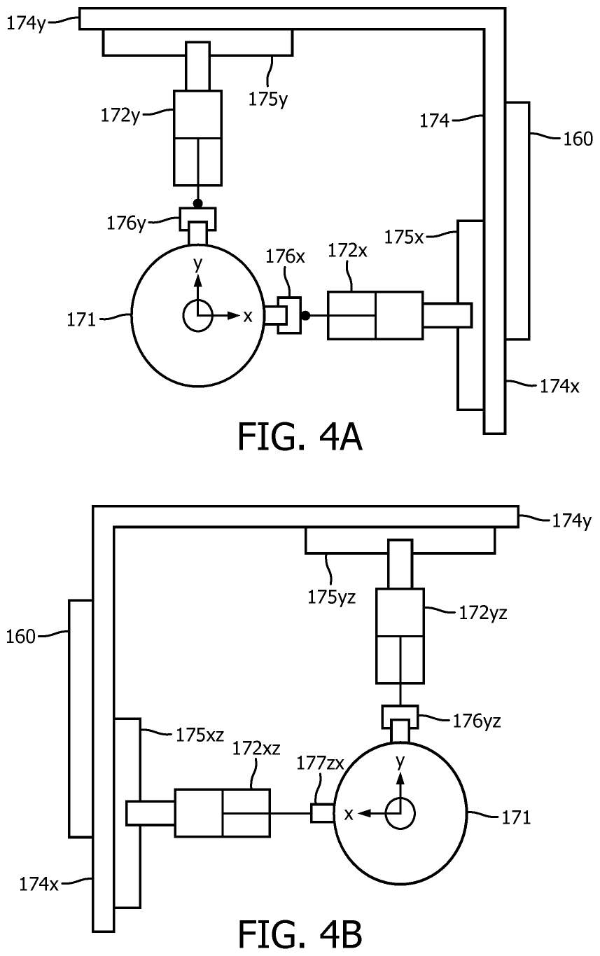

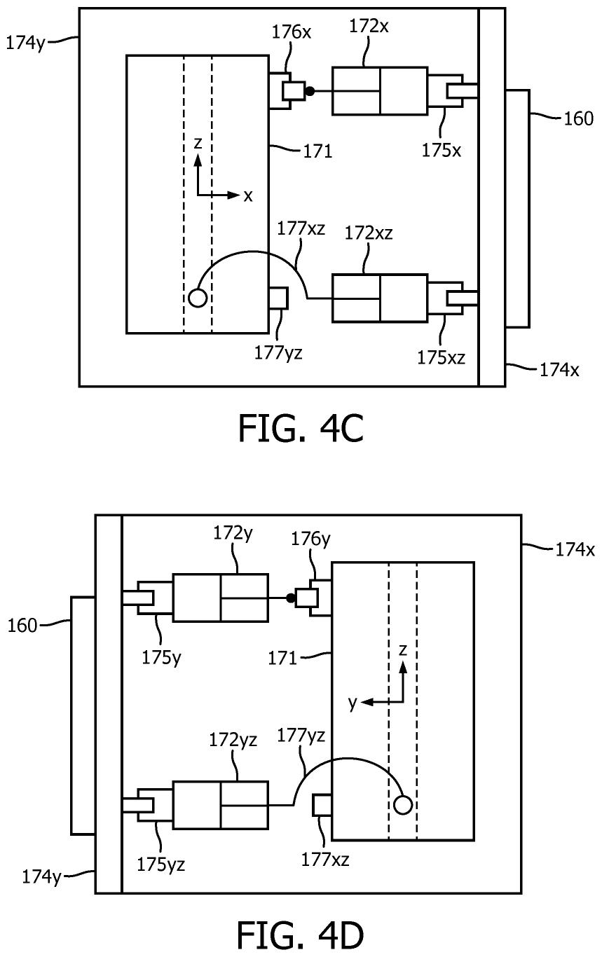

Referring to FIGS. 4A-4D, in one embodiment of compliant end-effector 70, a frame 174 includes a wall 174x and a wall 174y with a robotic arm 160 being adjoined to wall 174x.

This embodiment of compliant end-effector 70 further includes four (4) pressure actuator in the form of pneumatic pistons 172x, 172y, 172xz, and 172yz.

Pneumatic piston 172x is coupled to wall 174x via a slider joint 175x and is further coupled to a cylindrical tool holder 171 via a ball joint 176x to thereby effect a lateral motion and/or maintain a location of cylindrical tool holder 171 relative to wall 174x.

Pneumatic piston 172y is coupled to wall 174y via a slider joint 175y and is further coupled to a cylindrical tool holder 171 via a ball joint 176y to thereby effect a lateral motion and/or maintain a location of cylindrical tool holder 171 relative to wall 174y.

Pneumatic piston 172xz is coupled to wall 174x via a slider joint 175xz and is further coupled to a cylindrical tool holder 171 via a ball joint 176xz to thereby effect a rotational motion and/or maintain an orientation of cylindrical tool holder 171 relative to wall 174x.

Pneumatic piston 172y is coupled to wall 174y via a slider joint 175y and is further coupled to a cylindrical tool holder 171 via a ball joint 176y to thereby effect a rotational motion and/or maintain an orientation of cylindrical tool holder 171 relative to wall 174y.

Referring to FIGS. 3 and 4A-4D, pneumatic pistons 172x, 172y, 172xz, and 172yz are actuatable by robot actuation controller 80 via actuation signals 82 for controlling a manual positioning of tool holder 171 between a plurality of poses relative to frame 174, and pneumatic pistons 172x, 172y, 172xz, and 172yz may include a pressure sensor of any type for generating pressure signals 73 informative of a pressure setting of pneumatic pistons 172x, 172y, 172xz, and 172yz.

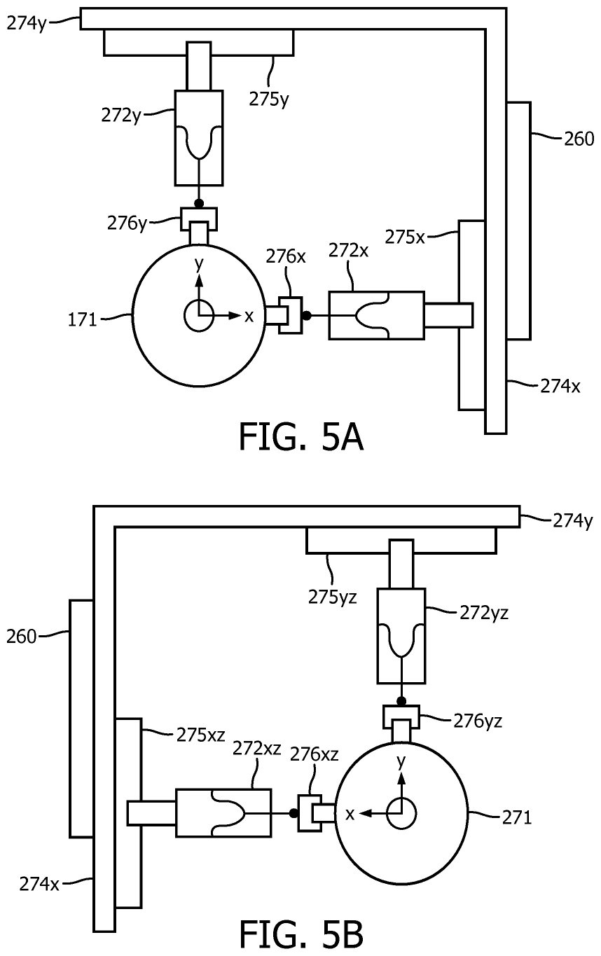

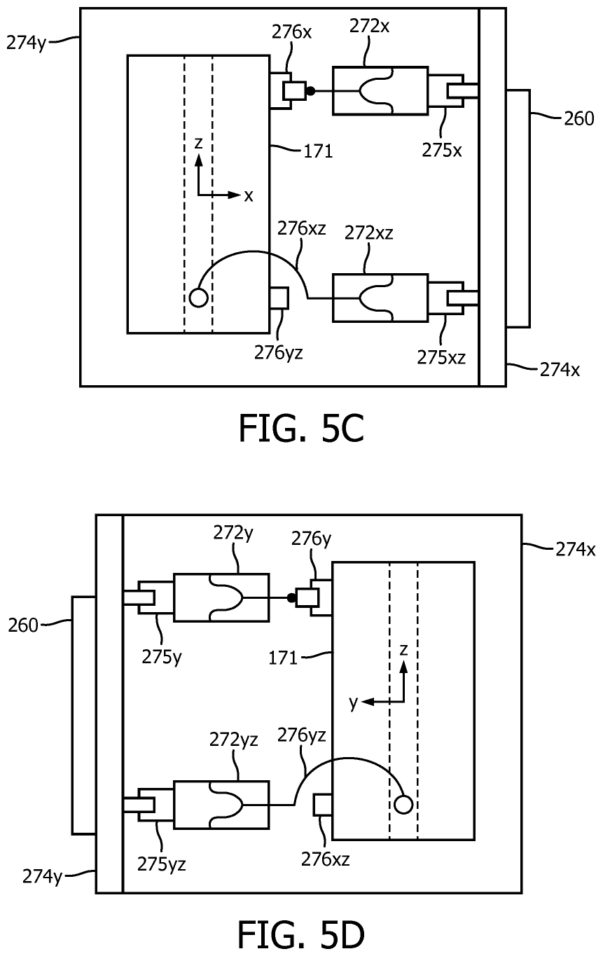

Referring to FIGS. 5A-5D, in a second embodiment of compliant end-effector 70, frame 274 includes a wall 274x and a wall 274y with a robotic arm 160 being adjoined to wall 274x.

This embodiment of compliant end-effector 70 further includes four (4) pressure actuator in the form of diaphragms 272x, 272y, 272xz, and 272yz.

Diaphragm 272x is coupled to wall 274x via a slider joint 275x and is further coupled to a cylindrical tool holder 271 via a ball joint 276x to thereby effect a lateral motion and/or maintain a location of cylindrical tool holder 271 relative to wall 274x.

Diaphragm 272y is coupled to wall 274y via a slider joint 275y and is further coupled to a cylindrical tool holder 271 via a ball joint 276y to thereby effect a lateral motion and/or maintain a location of cylindrical tool holder 271 relative to wall 274y.

Diaphragm 272xz is coupled to wall 274x via a slider joint 275xz and is further coupled to a cylindrical tool holder 271 via a ball joint 276xz to thereby effect a rotational motion and/or maintain an orientation of cylindrical tool holder 271 relative to wall 274x.

Diaphragm 272y is coupled to wall 274y via a slider joint 275y and is further coupled to a cylindrical tool holder 271 via a ball joint 276y to thereby effect a rotational motion and/or maintain an orientation of cylindrical tool holder 271 relative to wall 274y.

Referring to FIGS. 3 and 5A-5D, diaphragms 272x, 272y, 272xz, and 272yz are actuatable by robot actuation controller 80 via actuation signals 82 for controlling a manual positioning of tool holder 271 between a plurality of poses relative to frame 274, and diaphragms 272x, 272y, 272xz, and 272yz may include a pressure sensor of any type for generating pressure signals 73 informative of a pressure setting of diaphragms 272x, 272y, 272xz, and 272yz.

Referring to FIGS. 4 and 5, in practice, any type of actuator providing a mechanical resistance may be utilized in lieu of the pneumatic pistons and the diaphragms.

Referring back to FIG. 3, robot configuration controller 80 includes a global positioner 83, a target positioner 84 and a compliance profile 85 for performing two (2) main tasks of the present disclosure.

First, global positioner 83 processes end-effector positioning commands 90 informative of a desired robotic guidance of compliant end-effector 70 within the medical procedural space as known in the art of the present disclosure (e.g., image guided commands, user input commands, etc.) to thereby determine poses of linkage(s) 61 for robotically positioning compliant end-effector 70 in accordance with end-effector positioning commands 90. To this end, global positioner 83 generates actuation signal(s) 81 for actuating arm actuators(s) 62 to effect a motion and/or to maintain a position of linkage(s) 61 to thereby implement the determined poses of linkage(s) 61 for robotically positioning compliant end-effector 70 in accordance with end-effector positioning commands 90.

Second, target positioner 84 processes tool positioning commands 91 informative of a desired manual positionable range of tool holder 71 relative to robotic arm 60 (e.g., image guided commands, user input commands, etc.). To this end, target positioner 84 generates actuation signal(s) 82 for actuating tool actuators(s) 72 to effect a motion and/or to maintain a pose of tool holder 71 to thereby implement the desired manual positionable range of tool holder 71 relative to robotic arm 60. Specifically, target positioner 84 access a compliance profile 83 delineating actuation parameters of tool actuator(s) 72 for implementing the desired manual positionable range of tool holder 71 relative to robotic arm 60.

For example, referring to FIGS. 4A-4D, compliance profile 83 will delineate various pressure settings for pneumatic pistons 172 to limit a range of poses of tool holder 171 in compliance with the desired manual positionable range of tool holder 171 relative to robotic arm 160. More particularly, in practice, pistons 172 function as an air spring whereby, to obtain a certain compliance, the pressure in pistons 172 will be set to the desired value and then the command valve will be closed.

More particularly, pneumatic piston 172x will have a pressure setting for limiting any lateral motion of tool holder 171 along the x-axis between a minimum lateral displacement and a maximum lateral displacement established by the coupling of the pneumatic pistons 172 to frame 174.

Pneumatic piston 172y will have a pressure setting for limiting any lateral motion of tool holder 171 along the y-axis between a minimum lateral displacement and a maximum lateral displacement established by the coupling of the pneumatic pistons 172 to frame 174.

Pneumatic piston 172xz will have a pressure setting for limiting any rotational motion of tool holder 171 within the xz-plane between a minimum angular displacement and a maximum angular displacement established by the coupling of the pneumatic pistons 172 to frame 174.

Pneumatic piston 172yz will have a pressure setting for limiting any lateral motion of tool holder 171 within the yz-plane between a minimum angular displacement and a maximum angular displacement established by the coupling of the pneumatic pistons 172 to frame 174.

Also by example, referring to FIGS. 5A-5D, compliance profile 83 will delineate various pressure settings for diaphragms 272 to limit a range of poses of tool holder 271 in compliance with the desired manual positionable range of tool holder 271 relative to robotic arm 260.

More particularly, diaphragm 272x will have a pressure setting for limiting any lateral motion of tool holder 271 along the x-axis between a minimum lateral displacement and a maximum lateral displacement established by the coupling of the diaphragms 272 to frame 274.

Diaphragm 272y will have a pressure setting for limiting any lateral motion of tool holder 271 along the y-axis between a minimum lateral displacement and a maximum lateral displacement established by the coupling of the diaphragms 272 to frame 274.

Diaphragm 272xz will have a pressure setting for limiting any rotational motion of tool holder 271 within the xz-plane between a minimum angular displacement and a maximum angular displacement established by the coupling of the diaphragms 272 to frame 274.

Diaphragm 272yz will have a pressure setting for limiting any lateral motion of tool holder 271 within the yz-plane between a minimum angular displacement and a maximum angular displacement established by the coupling of the diaphragms 272 to frame 274.

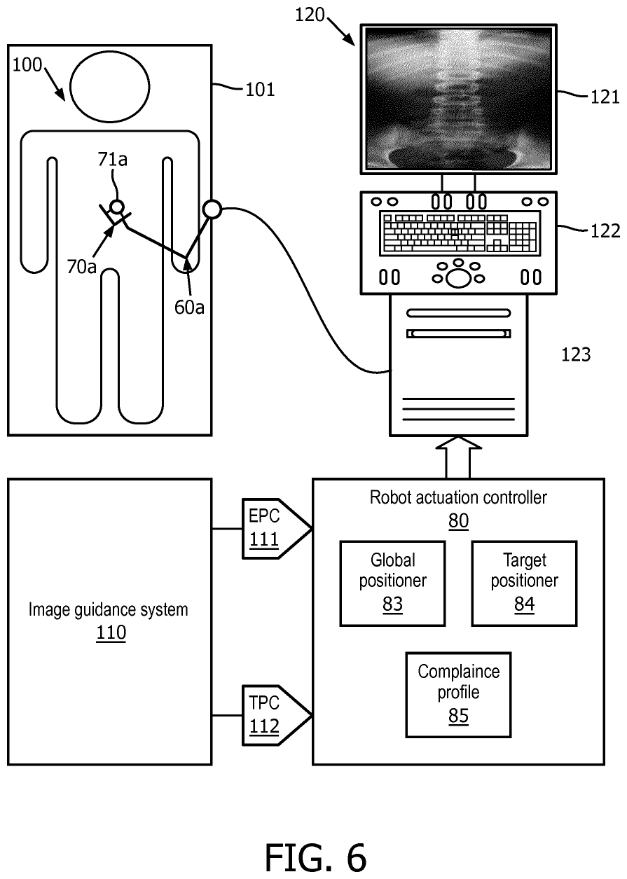

To facilitate a further understanding of the inventions of the present disclosure, the following description of FIGS. 6-8 teaches basic inventive principles of an exemplary embodiment of an image guided medical procedure system. From this description, those having ordinary skill in the art will appreciate how to apply the inventive principles of the present disclosure for making and using numerous and varied additional embodiments of an image guided medical procedure system of the present disclosure.

Referring to FIG. 6, robot actuation controller 80 (FIG. 3) is installed on a workstation 120 including a known arrangement of a monitor 121, a keyboard 122 and a computer 123 as known in the art of the present disclosure.

As installed, robot actuation controller 80 each may include a processor, a memory, a user interface, a network interface, and a storage interconnected via one or more system buses.

The processor may be any hardware device, as known in the art of the present disclosure or hereinafter conceived, capable of executing instructions stored in memory or storage or otherwise processing data. In a non-limiting example, the processor may include a microprocessor, field programmable gate array (FPGA), application-specific integrated circuit (ASIC), or other similar devices.

The memory may include various memories, as known in the art of the present disclosure or hereinafter conceived, including, but not limited to, L1, L2, or L3 cache or system memory. In a non-limiting example, the memory may include static random access memory (SRAM), dynamic RAM (DRAM), flash memory, read only memory (ROM), or other similar memory devices.

The user interface may include one or more devices, as known in the art of the present disclosure or hereinafter conceived, for enabling communication with a user such as an administrator. In a non-limiting example, the user interface may include a command line interface or graphical user interface that may be presented to a remote terminal via the network interface.

The network interface may include one or more devices, as known in the art of the present disclosure or hereinafter conceived, for enabling communication with other hardware devices. In an non-limiting example, the network interface may include a network interface card (MC) configured to communicate according to the Ethernet protocol. Additionally, the network interface may implement a TCP/IP stack for communication according to the TCP/IP protocols. Various alternative or additional hardware or configurations for the network interface will be apparent.

The storage may include one or more machine-readable storage media, as known in the art of the present disclosure or hereinafter conceived, including, but not limited to, read-only memory (ROM), random-access memory (RAM), magnetic disk storage media, optical storage media, flash-memory devices, or similar storage media. In various non-limiting embodiments, the storage may store instructions for execution by the processor or data upon with the processor may operate. For example, the storage may store a base operating system for controlling various basic operations of the hardware. The storage further stores global positioner 83 and target positioner 84 in the form of executable software/firmware.

Still referring to FIG. 6, an image guidance system 110 is provided to implement a medical procedure involving an actuation control by global positioner 83 for providing a robotic positioning end-effector 70a relative to an anatomical region of a patient 100 supported by table 101 and further involving an actuation control by target positioner 84 for providing a manual positioning of tool holder 71a relative robotic arm 60a.

In practice, image guidance system 110 includes one or more types of imaging modalities suitable for a medical procedure including, but not limited to, computed tomography imaging, magnetic resonance imaging, X-ray imaging and ultrasound imaging.

Also in practice, image guidance system 110 implements various image guidance techniques as known in the art of the present disclosure for commanding global positioner 83 via an end-effector command 110 to provide the robotic positioning end-effector 70a including, but not limited to, image segmentation, image registration, image path planning and image based robot servo control.

Further in practice, dependent upon the particular medical procedure, image guidance system 110 generates tool positioning commands 112 informative of a specification of a desired positionable range of tool holder 71a relative to robotic arm 60a in terms of an accuracy of the medical procedure and safety of patient 100.

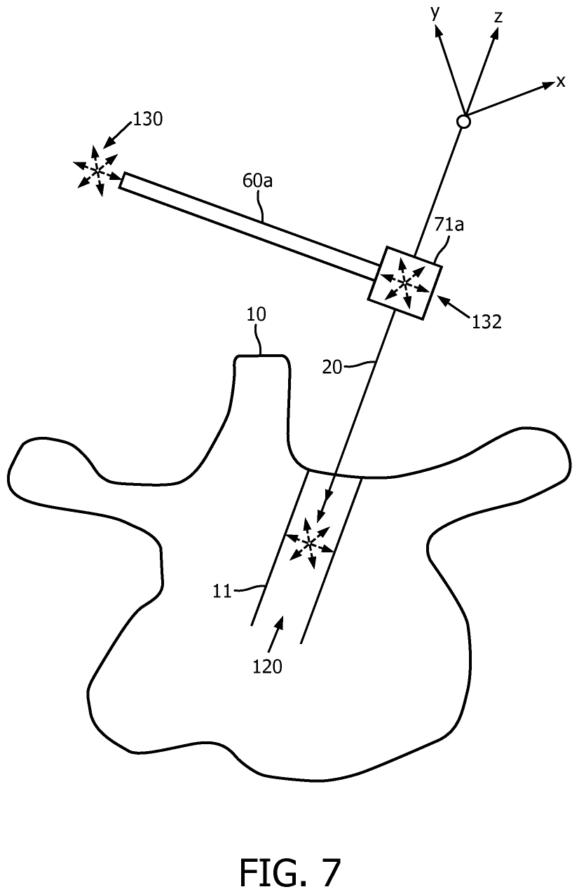

For example, referring to FIG. 7, upon the global positioning 130 of robotic arm 60a relative to a pedicle 10 during a pedicle screw replacement, target positioner 84 processes a tool position command 112 to ascertain actuation parameters from compliance profile 85 to set a desired positionable range 132 of tool holder 71a relative to robotic arm 60a that maintains a pedicle feeler 20 within a channel 11. More particularly, for the pneumatic piston and diaphragm embodiments, the actuation parameters would delineate pressure settings facilitating a uniform positionable range 132 of tool holder 71a relative to robotic arm 60a in all directions with displacement limits to thereby maintain the pedicle feeler 20 within channel 11.

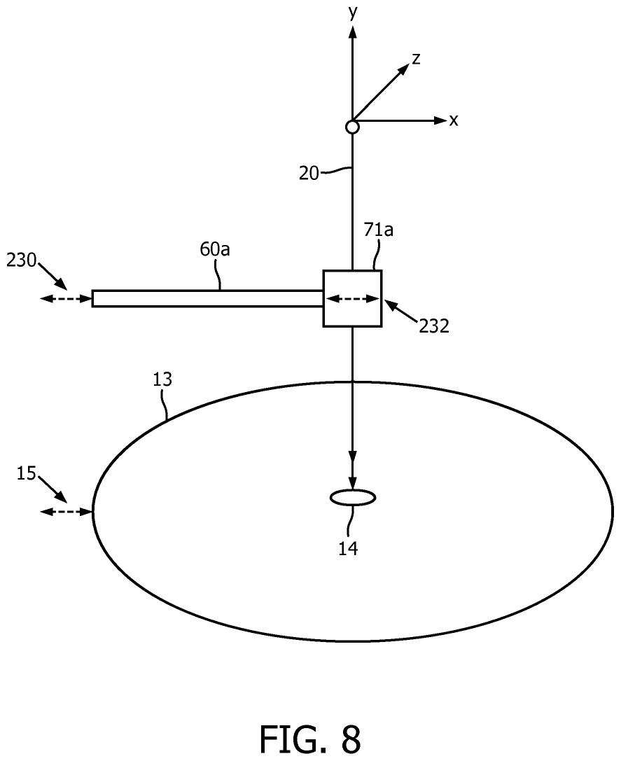

By further example, referring to FIG. 8, upon the global positioning 230 of robotic arm 60a relative to a kidney 13 during a kidney biopsy intervention, target positioner 84 processes a tool position command 112 to ascertain actuation parameters from compliance profile 85 to set a desired positionable range 232 of tool holder 71a relative to robotic arm 60a that moves with a respiration 15 of kidney 13. More particularly, for the pneumatic piston and diaphragm embodiments, the actuation parameters would delineate pressure settings facilitating a non-uniform positionable range 232 of tool holder 71a relative to robotic arm 60a with displacement limits to allows a clinician to chase target cancerous cells 14 in the direction of respiration 15 (e.g., a Superior-Inferior direction) yet constrain any displacement of tool holder 71a in directions orthogonal to respiration 15.

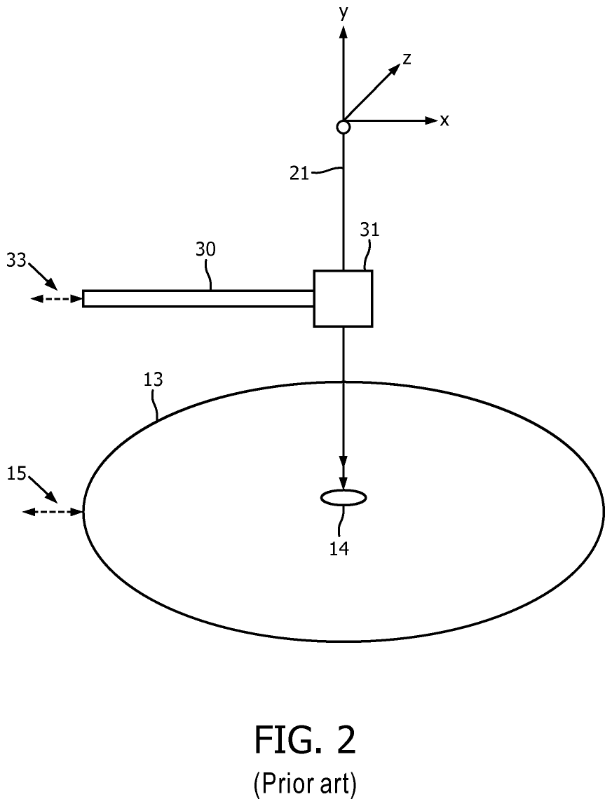

Referring to FIGS. 1-8, those having ordinary skill in the art of the present disclosure will appreciate numerous benefits of the inventions of the present disclosure including, but not limited to, systems, controllers and method for facilitating a manual adjustment of a tool-holder based on a targeted anatomy.

Furthermore, as one having ordinary skill in the art will appreciate in view of the teachings provided herein, features, elements, components, etc. described in the present disclosure/specification and/or depicted in the Figures may be implemented in various combinations of electronic components/circuitry, hardware, executable software and executable firmware and provide functions which may be combined in a single element or multiple elements. For example, the functions of the various features, elements, components, etc. shown/illustrated/depicted in the Figures can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, circuitry, combinations thereof, etc.) which is capable of (and/or configurable) to perform and/or control a process.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar function, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Furthermore, exemplary embodiments of the present disclosure can take the form of a computer program product or application module accessible from a computer-usable and/or computer-readable storage medium providing program code and/or instructions for use by or in connection with, e.g., a computer or any instruction execution system. In accordance with the present disclosure, a computer-usable or computer readable storage medium can be any apparatus that can, e.g., include, store, communicate, propagate or transport the program for use by or in connection with the instruction execution system, apparatus or device. Such exemplary medium can be, e.g., an electronic, magnetic, optical, electromagnetic, infrared or semiconductor system (or apparatus or device) or a propagation medium. Examples of a computer-readable medium include, e.g., a semiconductor or solid state memory, magnetic tape, a removable computer diskette, a random access memory (RAM), a read-only memory (ROM), flash (drive), a rigid magnetic disk and an optical disk. Current examples of optical disks include compact disk—read only memory (CD-ROM), compact disk—read/write (CD-R/W) and DVD. Further, it should be understood that any new computer-readable medium which may hereafter be developed should also be considered as computer-readable medium as may be used or referred to in accordance with exemplary embodiments of the present disclosure and disclosure.

Having described preferred and exemplary embodiments of novel and inventive compliant end-effectors and robot actuator controllers (which embodiments are intended to be illustrative and not limiting), it is noted that modifications and variations can be made by persons having ordinary skill in the art in light of the teachings provided herein, including the Figures. It is therefore to be understood that changes can be made in/to the preferred and exemplary embodiments of the present disclosure which are within the scope of the embodiments disclosed herein.

Moreover, it is contemplated that corresponding and/or related systems incorporating and/or implementing the device or such as may be used/implemented in a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

1. A compliant medical robot system, comprising:
a medical robot including a compliant end-effector adjoined to a robotic arm,
wherein the compliant end-effector includes at least one tool actuator adjoined to a tool holder to provide a manual positioning of the tool holder relative to the robotic arm; and
a robot actuator controller operable to be in communication with the at least one tool actuator,
wherein the robot actuator controller is configured to control an actuation of the at least one tool actuator based on a compliance profile delineating an actuation parameter to set a manual positionable range of the tool holder relative to the robotic arm in compliance with a tool positioning command specifying the manual positionable range of the tool holder relative to the robotic arm.

2. The compliant medical robot system of claim 1,
wherein the compliant end-effector further includes a frame adjoined to the robotic arm and the at least one tool actuator; and
wherein the actuation by the robot actuator controller of the at least one tool actuator in compliance with the tool position command includes:
the robot actuator controller further configured to actuate the at least one tool actuator to limit a range of poses of the tool holder relative to the frame.

3. The compliant medical robot system of claim 2,
wherein the compliant end-effector further includes at least one slider joint coupling the at least one actuator to the frame.

4. The compliant medical robot system of claim 2,
wherein the compliant end-effector further includes at least one ball joint coupling the at least one actuator to the tool holder.

5. The compliant medical robot system of claim 2,
wherein the compliant end-effector further includes at least one revolute joint coupling the at least one actuator to the tool holder.

6. The compliant medical robot system of claim 1,
wherein the at least one actuator includes a pressure actuator; and
wherein the compliant end-effector further includes a ball joint coupling the pressure actuator to the tool holder or further includes a revolute joint coupling the pressure actuator to the tool holder.

7. The compliant medical robot system of claim 6, wherein the pressure actuator is a pneumatic piston.

8. The compliant medical robot system of claim 6, wherein the pressure actuator is a diaphragm.

9. The compliant medical robot system of claim 6, wherein the compliant end-effector further includes:
a frame adjoined to the robotic arm; and
a slider joint coupling the pressure actuator to the frame.

10. The compliant medical robot system of claim 1,
wherein the at least one actuator includes a pressure actuator; and
wherein the compliant end-effector further includes a revolute joint coupling the pressure actuator to the tool holder.

11. The compliant medical robot system of claim 10, wherein the pressure actuator is a pneumatic piston.

12. The compliant medical robot system of claim 10, wherein the pressure actuator is a diaphragm.

13. The compliant medical robot system of claim 10, wherein the compliant end-effector further includes:
a frame adjoined to the robotic arm; and
a slider joint coupling the pressure actuator to the frame.

14. A robot actuator controller for a medical robot including a compliant end-effector adjoined to a robotic arm,
the robotic arm including at least one arm actuator to provide a robotic positioning of the compliant end-effector,
the compliant end-effector including at least one tool actuator coupled to a tool holder to provide a manual positioning of the tool holder,
the robot actuator controller comprising:
a global positioner operable to be in communication with the at least one arm actuator,
wherein the global positioner is configured to control an actuation of the at least one arm actuator to robotically position the compliant end-effector in compliance with an end-effector positioning command specifying a position of the compliant end-effector; and
a target positioner operable to be in communication with the at least one tool actuator,
wherein the target positioner is configured to control an actuation of the at least one tool actuator based on a compliance profile delineating an actuation parameter to set a manual positionable range of the tool holder relative to the robotic arm in compliance with a tool positioning command specifying the manual positionable range of the tool holder relative to the robotic arm.

15. The robot actuator controller of claim 14,
wherein the compliant end-effector further includes a frame adjoined to the robotic arm and the at least one tool actuator; and
wherein the actuation by the target positioner of the at least one tool actuator in compliance with the tool position command includes:
the target positioner further configured to actuate the at least one tool actuator to limit a range of poses of the tool holder relative to the frame.

16. A compliant medical robot control method for a medical robot including a compliant end-effector adjoined to a robotic arm,
the robotic arm including at least one arm actuator to provide a robotic positioning of the compliant end-effector,
the compliant end-effector including at least one tool actuator coupled to a tool holder to provide a manual positioning of the tool holder,
the compliant medical robot control method comprising:
a robot actuation controller controlling an actuation of the at least one arm actuator to robotically position the compliant end-effector in compliance with an end-effector positioning command specifying a position of the compliant end-effector; and
the robot actuation controller controlling an actuation of the at least one tool actuator delineating an actuation parameter to set a manual positionable range of the tool holder relative to the robotic arm in compliance with a tool positioning command specifying the manual positionable range of the tool holder relative to the robotic arm.

17. The compliant medical robot control method of claim 16,
wherein the compliant end-effector further includes a frame adjoined to the robotic arm and the at least one tool actuator; and
wherein the actuation by the robot actuation controller of the at least one tool actuator in compliance with the tool position command includes:
the target positioner further configured to actuate the at least one tool actuator to limit a range of poses of the tool holder relative to the frame.

18. The compliant medical robot control method of claim 17,
wherein the at least one tool actuator includes a pressure actuator; and
wherein the robot actuation controller controls a pressure setting of the pressure actuator to limit the range of poses of the tool holder relative to the frame.

19. The compliant medical robot control method of claim 18, wherein the pressure actuator is a pneumatic piston.

20. The compliant medical robot control method of claim 18, wherein the pressure actuator is a diaphragm.
